(19) United States
(12) Patent Application Publication
(10) Pub. No.: US 2015/0257983 A1
(43) Pub. Date: Sep. 17, 2015

(54) FLUORIDE-CONTAINING VARNISH FOR APPLICATION TO THE TOOTH SURFACE

(71) Applicant: Ivoclar Vivadent AG, Schaan (LI)

(72) Inventors: Urs Lendenmann, Grabs (CH); Carlo Bolis, Igis (CH)

(21) Appl. No.: 14/425,923

(22) PCT Filed: Sep. 3, 2013
(86) PCT No.: PCT/EP2013/068161

§ 371 Date: Mar. 4, 2015

(30) Foreign Application Priority Data
Sep. 7, 2012 (EP) 12183620.9

(51) Int. Cl. A61K 6/00

(52) U.S. Cl. CPC A61K 6/0017; A61K 6/007

(57) ABSTRACT

Fluoride-containing varnish for application to the tooth surface, which contains 25-87.5 wt. % organic solvent, 2-50 wt. % water, 5-50 wt. % water-insoluble film-forming agent, optionally 5-25 wt. % plasticizer, 0.5-10 wt. % inorganic fluoride source in dissolved form, in each case relative to the total weight of the varnish. The varnish is suitable in particular for use in the treatment of hypersensitive teeth and/or tooth necks, in the prophylaxis of caries, in the treatment of incipient caries lesions, the inhibition of demineralization and/or tooth erosion.

The invention relates to a fluoride-containing varnish for application to the tooth surface, which is suitable in particular for use in the treatment of hypersensitive teeth and/or tooth necks, in the prophylaxis of caries, in the treatment of incipient caries lesions, the inhibition of demineralization and/or tooth erosion.

In the last decades fluorides have led to an impressive decrease in caries diseases in children. While the caries-reducing action of fluorides in children's permanent teeth has been clearly demonstrated clinically, this success of fluoride is unfortunately accompanied by more frequent reports of fluorosis, particularly if fluoride is administered as a food supplement or via drinking water. Fluorosis can already occur to an increased extent at fluoride concentrations in drinking water of greater than 1 ppm. For children under 6 years of age, exclusively fluoride varnishes are recommended for fluoridation by the American Dental Association, in order to keep the risk of fluoride poisoning by swallowing low.

Fluoride varnishes for targeted, local application for prophylaxis of caries have already been on the market for years. The prophylactic action on caries is based on fluoridation of the tooth, more accurately the deposition of a calcium fluoride-like layer on the tooth and an increase in the fluoride content in the topmost layers of enamel. As a result, dissolving of the tooth enamel, which chiefly consists of hydroxyapatite, under attack by acid is slowed down. Fluoride also speeds up the crystallization of hydroxyapatite. Slightly demineralized enamel already requires considerably higher fluoride concentrations in the surrounding medium, compared with healthy enamel, in order to prevent further demineralization.

Fluoride varnishes currently on the market which do not require photopolymerization are chiefly suspensions of sodium fluoride in a solution of natural resins.

The product Duraphat® (Colgate) contains various resins, such as colophony, mastic and shellac, in addition to sodium fluoride. Ethanol serves as the solvent. It has been found that Duraphat® is active in the prevention of root caries, for treatment of hypersensitive tooth necks and for slowing down the rate of progression of erosive tooth changes. The accumulation of fluoride depends on the application time. A six-hour application led to a considerably higher fluoride enrichment than a one-hour application (Hellwig, Oralprophylaxe & Kinderzahnheilkunde 30 (2008) 4, 139-143).

Clinpro® White Varnish (3M-ESPE) contains 5% sodium fluoride and tricalcium phosphate (TCP) in an alcoholic solution of modified resins. The resins are white or tooth-coloured, with the result that the product is practically invisible after application. In vitro, the release of fluoride is said to last for a period of 24 hours.

MI Varnish® (GC Corp.) contains, in addition to 5% sodium fluoride, hydrogenated colophony and CPP-ACP (casein phosphopeptide-amorphous calcium phosphate) in a solution of polyvinyl acetate in ethyl alcohol. It supplies the tooth surface with bioavailable calcium, phosphate and fluoride.

In addition, suspensions of sodium fluoride and calcium fluoride in a solution of nitrocellulose in a mixture of ethyl acetate and isopentyl propionate (Bifluorid 10, Bifluorid 12; VOCO) are used for caries prevention and treatment of hypersensitivity.

The product Fluor Protector® (Ivoclar-Vivadent) contains a mixture of a fluorosilane and a polyisocyanate dissolved in a mixture of ethyl acetate and isoamyl propionate.

The known products are not satisfactory in all respects. Suspended fluoride can settle during storage, with the result that suspensions often have to be homogenized by mixing before application. After application, the suspended fluoride furthermore must first dissolve, in order that it can interact with the hydroxyapatite of the tooth, which delays the release of fluoride. A wide particle size distribution of the fluoride particles can moreover result in an irregular release of fluoride.

The fluoride source fluorosilane is indeed soluble in organic solvents, but the initial release of fluoride is delayed because the fluorosilane must first be hydrolysed.

The varnishes are usually applied to the tooth surface with a small brush or a small sponge. It is advantageous if a thin film can be produced, which is found by the patient to be the least possible trouble after the treatment. However, many suspensions are high-viscosity liquids which result in relatively thick films.

The high sodium fluoride concentrations necessary for adequate fluoridation increase the risk of fluorosis, especially with a high viscosity of the varnishes and the resulting thick varnish films which therefore contain a large amount of fluoride, since the varnish films which become detached over time are often swallowed.

Natural resins such as colophony, colophony derivatives, mastic or shellac are pale yellow to amber-coloured and bring about an intensive colouring of the varnishes. The varnish films are therefore visible on the tooth surface during the wearing time after application, which is found to be troublesome by many patients. Although the suspended sodium fluoride brings about a brightening of the varnish film, this happens at the expense of transparency, i.e. the suspension and also the dried film have an opaque effect and contrast visually with the tooth surface. Colophony can trigger allergic reactions and asthma and cause eczema.

Other varnish components, such as polyisocyanates, react readily with water to give polyureas, which necessitates an expensive water-tight packaging for multiple uses. In addition, the tooth surface must be dried thoroughly before the application, which is not easy to achieve especially in the case of children.

Solvents such as ethyl acetate and isoamyl propionate have a strong smell which is often found to be unpleasant.

The object of the invention is to provide a fluoride-containing varnish for tooth treatment which does not have the disadvantages described above. In particular, the varnish is to make possible an effective fluoridation and remineralization of the tooth enamel at a low fluoride concentration. The varnish is to have the lowest possible viscosity and allow the production of colourless varnish films.

According to the invention, this object is achieved by fluoride-containing varnishes which contain
25-87.5 wt. % organic solvent,
2-50 wt. % water,
5-50 wt. % water-insoluble film-forming agent,
0.5-10 wt. % inorganic fluoride source in dissolved form
and preferably
5-25 wt. % plasticizer,

in each case relative to the total weight of the varnish.

Preferred varnishes are those which contain
45-80 wt. %, in particular 48-70 wt. % organic solvent,
5-25 wt. %, in particular 10-20 wt. % water,
5-25 wt. %, in particular 8-20 wt. % water-insoluble film-forming agent,
5-22 wt. %, in particular 10-20 wt. % plasticizer and
0.5-5 wt. %, in particular 1-3 wt. % inorganic fluoride source in dissolved form.

It has been found, surprisingly, that the varnishes according to the invention show a high release of fluoride even at relatively low amounts of fluoride, and in particular that the fluoride released is absorbed very effectively by tooth enamel.

Products according to the state of the art in which the fluoride source (mostly sodium fluoride) is present as a suspended powder must remain on the tooth for a long time, since the fluoride must first dissolve before it can be absorbed by the tooth enamel. The dissolving of the fluoride is effected by water from the saliva. Since known varnishes are usually anhydrous and hydrophobic, the saliva comes into contact with the varnish layer and dissolves out fluoride only on the surface. It is a further problem that the fluoride ions released can diffuse through the hydrophobic varnish film in the direction of the tooth surface only with difficulty, and therefore mostly migrate into the oral cavity. This means that although known products can release large amounts of fluoride, only a little fluoride reaches the enamel.

Plasticizers which are preferred according to the invention for the preparation of the varnishes are fatty alcohols, polyethylene glycol (PEG), polypropylene glycol (PPG), dexpanthenol and, preferably, esters, such as sugar esters and alkyl or phenyl esters of di- or tricarboxylic acids or hydroxydi- or -tricarboxylic acids having one or more hydroxyl groups.

Preferred esters are, for example, alkyl or phenyl esters (C₁₂ and higher, preferably C₁₂₋₂₄) of dicarboxylic acids, hydroxydicarboxylic acids having one (preferably diisostearyl malate, obtainable e.g. from Lubrizol under the name Schercemol™ DISM Ester) or more hydroxyl groups; alkyl or phenyl esters of polyethylene glycols or polypropylene glycol or combinations thereof (preferably PEG/PPG-8/3 diisostearate, obtainable e.g. from Lubrizol under the name Hydramol™ PGPD Ester); alkyl or phenyl esters of glycerol, di- or triglycerol (preferably polyglyceryl-3 laurate, obtainable e.g. from Lubrizol under the name Hydramol™ TGL Ester); alkyl or phenyl esters of di- and tricarboxylic acids or hydroxydi- and -tricarboxylic acids with Guerbet alcohols (C₁₆ and higher, preferably C₁₆₋₄₀), preferably trioctyldodecyl citrate, adipic acid polyesters (obtainable e.g. from Lanxess under the name Ultramoll®), alkylsulphonic acid phenyl esters (obtainable e.g. from Lanxess under the name Mesamoll®), esters of hydrogenated colophony, fatty alcohols, dexpanthenol, polyethylene glycol (PEG), polypropylene glycol (PPG).

Sugar esters are particularly suitable as the plasticizer, in particular esters of an organic acid, preferably a monocarboxylic acid having 1 to 18, in particular 1 to 4 carbon atoms, with a mono- or disaccharide, wherein those esters in which the hydroxyl groups of the sugar component are esterified completely are particularly preferred. Esters which are very particularly preferred are those of sucrose with acetic acid or isobutyric acid or mixed esters of sucrose with acetic acid and isobutyric acid, and in particular sucrose acetoisobutyrate (SAIB; INCI name: sucrose acetate isobutyrate). SAIB is a mixture of different isomers, the composition of which approximately corresponds to sucrose diacetate hexaisobutyrate. It is a high-viscosity liquid. SAIB is an emulsifier and foodstuffs additive (E number 444).

The varnishes according to the invention are characterized in that they contain water. Varnishes which contain a mixture of water and a water-miscible organic solvent are very particularly preferred. Preferred water-miscible organic solvents are mono- or polyvalent C₂-C₄ alcohols or C₂-C₄ ketones and mixtures thereof, particularly preferably isopropanol, acetone and very particularly preferably ethanol.

Varnishes which are preferred according to the invention contain ethanol, wherein the ratio of ethanol and water is such that the mixture contains 10 to 30 wt. % water and 70 to 90 wt. % ethanol, in each case relative to the total amount of ethanol and water.

According to one embodiment, the varnishes according to the invention preferably contain no unpleasantly smelling solvents, such as e.g. ethyl acetate, isoamyl propionate, isopentyl propionate and alkanes, such as C₅-C₁₂ alkanes, for example n-hexane.

Inorganic fluorides, bifluorides (hydrogen difluorides) or fluorine complex salts are preferably used as the fluoride source, particularly preferably NH₄F, KF, RbF, CsF, NH₄HF₂, KHF₂, sodium hexafluorophosphate, very particularly preferably NH₄F and KF. A mixture of two or more of these substances can also be used as the fluoride source. For simplicity, hereinafter the fluoride source is also called fluoride.

The fluoride source is to be readily soluble in water, i.e. have a solubility at 25° C. of at least 5 mol in 1 kg water, preferably at least 10 mol in 1 kg water. Fluorides which are particularly suitable according to the invention and solubilities thereof are as follows:

| | Solubility | Solubility |
|---|---|---|
| Fluoride source | (g/100 g H₂O) | (mol/kg H₂O) |
| Potassium bifluoride; KHF₂ | 39.2 | 5.0 |
| Sodium hexafluorophosphate | 103 | 5.5 |
| monohydrate; NaPF₆ x H₂O | | |
| Ammonium bifluoride; NH₄HF₂ | 60.2 | 10.6 |
| Silver(I) fluoride; AgF | 172 | 13.6 |
| Potassium fluoride; KF | 102 | 17.6 |
| Ammonium fluoride; NH₄F | 83.5 | 22.5 |
| Rubidium fluoride; RbF | 300 | 28.7 |
| Caesium fluoride; CsF | 573 | 37.7 |

The varnishes according to the invention have the advantage that the fluoride source is already present in the varnish in dissolved form, with the result that no delays occur in the release of fluoride due to dissolving processes. The amount of dissolved fluoride can be controlled by the nature of the fluoride source, the nature and amount of the solvent and the water content of the varnish.

The fluoride-containing varnishes according to the invention preferably contain, as the film-forming agent, an alcohol- or ketone-soluble, preferably an ethanol-soluble, polymer. The term alcohol- or ketone-soluble relates to the abovementioned solvents. The film-forming agent is insoluble in water.

Polymers which have a solubility in ethanol or mixtures of ethanol and up to 50 wt. % water of at least 6 wt. % and preferably at least 10 wt. % are preferred. By water-insoluble polymers are meant those substances which have a solubility in water of at most 5 wt. %, in particular at most 4 wt. %. Unless stated otherwise, all the solubility data herein are based on a temperature of 25° C.

Suitable film-forming agents are neutral, cationic and in particular anionic film-forming polymers.

Examples of anionic polymers are homo- and copolymers of acrylic acid and methacrylic acid, copolymers of acrylic acid, acrylates and acrylamide, and homo- and copolymers of acrylic acid esters and methacrylic acid esters.

Particularly suitable polymers are copolymers of t-butyl acrylate, ethyl acrylate, methacrylic acid (e.g. Luvimer® 100P), copolymers of ethyl acrylate and methacrylic acid (e.g. Luviflex® Soft and Luvimer® MAE), copolymers of N-tert-butylacrylamide, ethyl acrylate, acrylic acid (Ultrahold® 8 strong from BASF AG), copolymers of vinyl acetate, crotonic acid and optionally further vinyl esters (e.g. Luviset®, BASF AG), anionic polysiloxanes, e.g. carboxyfunctional, t-butyl acrylate, methacrylic acid (e.g. Luviskol® VBM, BASF AG). Examples of anionic polymers are furthermore vinyl acetate/crotonic acid copolymers, such as are commercially available, for example, under the names Resyn® (Akzo Nobel) and Gafset® (GAF).

The group of polymers which are suitable according to the invention as the film-forming agent furthermore includes Balance® CR (Akzo Nobel; acrylate copolymer), Balance® 0/55 (Akzo Nobel; acrylate copolymer), Balance® 47 (Akzo Nobel; octylacrylamide/acrylate/butylaminoethyl methacrylate copolymer), Amphomer® HC (Akzo Nobel; acrylate/octylacrylamide copolymer), Amphomer® 28-4910 (Akzo Nobel; octylacrylamide/acrylate/butylaminoethyl methacrylate copolymer), Advantage® Plus (lSP; VA/butyl maleate/isobornyl acrylate copolymer), Luviflex® Silk (BASF; reaction product of t-butyl acrylate, methacrylic acid and dimethicone copolyol), Resyn XP (Akzo Nobel; acrylates/octylacrylamide copolymer).

Suitable polymers are also amphoteric polymers, such as the octylacrylamide/methyl methacrylate/tert-butylaminoethyl methacrylate/2-hydroxypropyl methacrylate copolymers obtainable under the name Amphomer® (Akzo Nobel).

Particularly preferred anionic film-forming agents are acrylate and methacrylate homopolymers, acrylate and methacrylate copolymers, terpolymers of C₄-C₈ alkylacrylamide, acrylates and acrylic acid, terpolymers of methacrylic acid and acrylates, vinyl acetate/crotonic acid copolymers and mixtures thereof. Very particularly preferred film-forming agents are terpolymers of tert-butyl acrylate, ethyl acrylate and methacrylic acid (INCI name: acrylates copolymer).

The polymers are preferably used in the ionic and not in the neutralized form.

It has been found, surprisingly, that with the varnishes according to the invention, and in particular varnishes which contain a plasticizer, a high fluoride content is already achieved on the tooth enamel after a short time. It is assumed that after drying of the varnishes under intraoral conditions, sufficient water remains in the film to keep the dissolved fluoride completely or partly in solution. The organic solvent contained in the varnishes evaporates during drying faster than the water content, with the result that, in spite of the concentrating operation, the fluoride source is still present at least partly in dissolved form even after the drying. The plasticizer has a positive effect on the residual water content of the film. The water which remains in the varnish film allows a rapid diffusion of the fluoride ions in the direction of the tooth enamel, with the result that a high fluoride content is rapidly achieved on the tooth enamel.

During curing of the varnish on the teeth, the plasticizer moreover prevents the film-forming agent from drying out or precipitating inhomogeneously, and prevents crystalline domains of the film-forming agent which are too large from forming.

It is particularly advantageous that after topical application to the tooth enamel the varnishes according to the invention already lead to a significant increase in the fluoride concentration on and in the enamel after a short time. For superficial, alkali-soluble fluoride (ASF; “on enamel”), values of from 10 to 50 μg/cm² are achieved after a treatment time of one hour, and for structurally bonded fluoride (SBF; “in the enamel”) values of from 5 to 20 μg/cm². Structurally bonded fluoride is a fluoride which is incorporated into the hydroxyapatite crystals of the tooth enamel, and can be determined after etching of the enamel surface with HClO₄. The determination of the fluoride on the enamel and in the enamel is described in Example 2. The rapid increase in the fluoride concentration on and in the enamel makes possible a treatment time of 1 hour or less, preferably 0.5 to 1 hour.

Varnishes which result in a fluoridation of the enamel (ASF) of at least 10 μg/cm², in particular 10 to 50 μg/cm², preferably at least 15 μg/cm², in particular 15 to 50 μg/cm², measured on bovine tooth enamel analogously to Example 2, after a treatment time of one hour are preferred.

In addition to the components mentioned, the varnishes according to the invention can contain conventional additives, such as flavouring substances, sweetening agents, fillers and antibacterial active ingredients. These substances are conventionally used in amounts of in each case 0.01 to 10 wt. %, preferably 0.01 to 5 wt. %, particularly preferably 0.01 to 2 wt. %.

Suitable flavouring substances are natural and artificial flavourings or flavouring extracts with a smell/taste of peppermint, orange, strawberry, vanilla, melon etc. Suitable flavourings are listed in “COMMISSION DECISION of 23 Feb. 1999 adopting a register of flavouring substances used in or on foodstuffs” drawn up in application of Regulation (EC) No. 2232/96 of the European Parliament and of the Council of 28 Oct. 1996 (1999/217/EC).

Suitable sweetening agents are, in particular, natural and synthetic sugar substitutes, such as sugar alcohols (sorbitol, mannitol, xylitol etc.) and sweeteners (saccharin, acesulfame, aspartame, cyclamate, neohesperidin, sucralose, stevioside etc.)

Suitable fillers are, in particular, precipitated or pyrogenic silicic acids (e.g. Aerosil from Evonik), nanoscale metal oxides, such as e.g. pyrogenic aluminium oxide (e.g. Aeroxid Alu C, Evonik) or mineral fillers, such as e.g. sheet silicates.

Suitable antibacterial active ingredients are, in particular, peroxides, such as hydrogen peroxide or carbamide peroxide, furthermore chlorohexidine, cetylpyridinium chloride, hydroxybenzoic acid esters (parabens) etc.

Varnishes of this invention can also contain further inorganic substances, for example mineral sources, which promote the remineralization of teeth. Examples are amorphous calcium phosphate, tricalcium phosphate, calcium carbonate, hydroxyapatite, fluorapatite, potassium phosphate etc. These substances are ideally present in such a fine form that they remain suspended in a low-viscosity liquid. Finely ground or nanoscale particle sizes of such substances are therefore desired. Particle sizes of from 0.005 μm to 10 μm are preferred, particularly preferably 0.005 μm to 1 μm and very particularly preferably 5 nm to 200 nm.

Those varnishes which contain or consist of a combination of preferred and in particular of particularly preferred components are of course particularly advantageous. Such combinations are therefore preferred.

A very particularly advantageous combination is, for example, a varnish which contains
50-70 wt. % ethanol,
10-20 wt. % water,
5-15 wt. % terpolymer of tert-butyl acrylate, ethyl acrylate and methacrylic acid (INCI: acrylates copolymer),
10-20 wt. % sucrose acetate isobutyrate,
1-2 wt. % ammonium fluoride,
0.05-0.2 wt. % flavouring substance(s) and
0.01-0.1 wt. % sweetening agent.

A preferred fluoride varnish without plasticizer is:
60-75 wt. % ethanol
7.5-20 wt. % water
10-25 wt. % terpolymer of tert-butyl acrylate, ethyl acrylate and methacrylic acid (INCI: acrylates copolymer)
0.01-0.1 wt. % sweetening agent
1-2 wt. % ammonium fluoride
0.1-0.5 wt. % pyrogenic aluminium oxide
0.05-0.2 wt. % flavouring substance(s).

The varnishes according to the invention can be rapidly applied to the tooth with a brush or small sponge and, after evaporation of the solvents, form a colourless, clear and firmly adhering varnish film there, which shows a rapid and high release of fluoride and already results in a large amount of alkali-soluble fluoride on the enamel surface after a short application time of one hour. The varnishes result in a calcium fluoride-like layer on the enamel and enrich the enamel underneath the surface with fluoride. Astonishingly, fluoridation of the topmost enamel layer at least equivalent to more highly concentrated products is produced.

The varnishes according to the invention are characterized by a combination of advantageous properties. They show a high initial release of fluoride and form clear and firmly adhering varnish films after drying. Furthermore, the varnishes have a very low viscosity. The viscosity is 10 to 5,000 mPa·s, preferably 10 to 1,000 mPa·s and particularly preferably 10 to 250 mPa·s (measured with a cone-plate measuring system at 15.0° C. and a shear rate of 100 s⁻¹).

The varnishes according to the invention are suitable in particular for use in the treatment of hypersensitive teeth and/or tooth necks, in the prophylaxis of caries, in the treatment of incipient caries lesions, the inhibition of demineralization of teeth and/or tooth erosion, for protection of fissures and pits and for fluoridation of enamel.

The invention is explained in more detail in the following with the aid of figures and examples.

### EMBODIMENT EXAMPLES

### Example 1

Preparation of a Fluoride-Containing Varnish

A varnish having the following composition was prepared by mixing the components:

| Proportion | Component |
|---|---|
| 58.70% | ethyl alcohol puriss. |
| 14.68% | deionized water |
| 15.00% | sucrose acetate isobutyrate (SAIB) |
| 10.00% | acrylate copolymer (Luvimer 100P, BASF) |
| 1.50% | ammonium fluoride |
| 0.10% | synth. peppermint flavouring |
| 0.02% | sweetening agent (saccharin) |

All the constituents were dissolved, accompanied by stirring, in the solvent introduced. A stable, practically colourless, homogeneous, low-viscosity varnish was obtained.

### Example 2

In Vitro Fluoridation of Enamel by Fluoride Varnishes (24 h)

For determination of the in vitro fluoridation, cylindrical blocks of enamel (n=18) were drilled out of bovine teeth and polished (SiC, 4,000 grit). Thereafter, the enamel samples were demineralized with lactic acid at room temperature for 1 hour (demineralization solution in accordance with Zahradnik et al., J Dent Res, 1976, 55; 4; 664-670, but with a lower calcium concentration than in saliva: lactic acid 0.1 mol/l; Ca₃(PO₄)₂ 50 mg/l; NaN₃ 0.02% (preservative); adjust to pH 4.4 with 2-5 M KOH), and were then coated, in accordance with the manufacturer's instructions, with the varnishes to be tested and were dried (25° C., 1 h). The varnish according to the invention was applied to the enamel with a small brush (Microbrush®) and dried (25° C., 1 h). The dried samples were stored in artificial saliva (37° C., 1 h or 24 h). The artificial saliva was replaced after one hour in order to avoid the formation of higher fluoride concentrations in the elution medium. The varnishes were then removed with ethanol or acetone. Superficial, alkali-soluble fluoride (ASF) and structurally bonded fluoride (SBF) were extracted and measured with an ion-selective electrode. A non-paired t-test was performed for the statistical analysis, p<0.05.

Artificial saliva was prepared in accordance with Takagi, Caries Res 1992; 26; 321-327, but without fluoride. Composition: 1.2 mM CaCl₂ 2 H₂O, 0.72 mM KH₂PO₄, 30 mM KCl, 50 mM HEPES (=4-(2-hydroxyethyl)-1-piperazine-ethane-sulphonic acid), pH 7.

The determination of the superficial, alkali-soluble fluoride

(ASF) was carried out in accordance with Caslayska et al., Archs oral Biol 20; 333-339, 1975. For this, the superficial, CaF₂-like fluoride was dissolved by immersion of the sample for 24 hours into 1 M KOH, the solution was then neutralized with HNO₃ and, after addition of a buffer to adjust the total ionic strength (TISAB-II buffer, preparation: dissolve 58 g NaCl in 500 g deion. H₂O in a 1,000-ml glass beaker, add 57 ml glacial acetic acid and 4(1,2-g CDTA cyclohexylenediaminetetraacetic acid), accompanied by stirring, and dissolve; thereafter adjust to a pH of 5-5.5 with 5 M NaOH (control with pH electrode), allow the solution to cool and top up to 1,000 ml with deion. H₂O), the fluoride concentration was measured with a fluoride electrode.

For determination of the structurally bonded fluoride (SBF), a biopsy was performed in accordance with Sieck et al., J Dent Res 69; 1261-1265, 1990. The surface of the enamel samples was etched with 0.5 M HClO₄ for 1 hour. About 100 μm of the surface layer was dissolved in the process. After neutralization and addition of TISAB-II, the fluoride concentration was measured again with a fluoride electrode. The results are shown in Table 1.

TABLE 1
Results of the in vitro fluoridation of enamel after
24 h of treatment/storage in artificial saliva

| | | Fluoride conc. | ASF | SBF |
|---|---|---|---|---|
| Product | Manufacturer | [ppm] | [μg/cm²] | [μg/cm²] |
| Example 1 | | 7,700 | 20.4 ± 6.2^{a} | 17.9 ± 3.6^{c} |
| Duraphat*) | Colgate | 22,600 | 19.3 ± 7.6^{a} | 21.0 ± 6.9^{a} |
| Clinpro White | 3M-ESPE | 22,600 | 9.3 ± 2.4^{b} | 11.5 ± 1.8^{b} |
| Varnish*) | | | | |
| Profluorid | VOCO | 22,600 | 11.6 ± 2.9^{c} | 11.9 ± 2.7^{b} |
| Varnish*) | | | | |
| Control (water) | | <1 | 1.4 ± 0.8^{d} | 4.2 ± 2.5^{d} |

*)Comparative example
Values with the same superscript letters do not differ significantly

After application of the varnishes to the tooth enamel, ASF and SBF were increased significantly, compared with water, in all the samples.

In spite of a significantly lower fluoride content, the varnish according to the invention achieved peak values in the fluoride concentration on (ASF) and in (SBF) the enamel after topical application.

### Example 3

EDX Investigation of the Fluoridation of Enamel

The fluoridation of enamel surfaces by various fluoride varnishes was investigated by energy-dispersive x-ray analysis (EDX). The varnishes investigated are shown in Table 2.

For determination of the fluoridation of enamel, cylindrical test pieces having a diameter of 4 mm were drilled out of ground and polished (SiC, 4,000 grit) bovine teeth and were demineralized (1 h in 0.1 M lactic acid, adjusted to pH 4.4).

TABLE 2
Fluoride varnishes investigated

| | | | | Conc. of |
|---|---|---|---|---|
| | | Man- | Fluoride | fluoride |
| Product | Batch | ufacturer | source | [ppm] |
| Example 1 | | | NH₄F | 7,700 |
| Fluor Protector*) | PL1004 | Ivoclar- | fluorosilane | 1,000 |
| | | Vivadent | | |
| Duraphat*) | 116416 | Colgate | NaF | 22,600 |
| MI Varnish*) | 1110211 | GC Corp. | NaF | 22,600 |
| Clinpro White | M14300H1C | 3M-ESPE | NaF | 22,600 |
| Varnish*) | | | | |
| Profluorid Varnish*) | 1114277 | VOCO | NaF | 22,600 |

*)Comparative example

The demineralized enamel surfaces, with the exception of the negative control, were treated with the corresponding fluoride varnish (leave to dry on for 5 min.) and thereafter stored in artificial saliva for one hour at 37° C. After removal from the artificial saliva, the varnish was removed by swirling the test piece in pure ethanol (Example 1) or acetone (remaining varnishes) and the test piece was rinsed briefly with water. After drying of the test pieces with compressed air, the enamel surface was investigated by means of EDX (spot size approx. 100 μm). The results are summarized in Table 3.

The results of the investigation show that different fluoride contents in the superficial enamel can be measured by EDX. The sample treated with varnish from Example 1 has by far the highest fluorine content. The present investigation confirms the very good fluoridation action of the varnish from Example 1 on demineralized enamel already after a short action time.

TABLE 3
Results of the EDX measurements

| Product | C | F | Na₂O | MgO | P₂O₅ | Cl | K₂O | CaO |
|---|---|---|---|---|---|---|---|---|
| Example 1 | 9.27 | 4.15 | 0.89 | 0.31 | 36.49 | 0.00 | 0.56 | 48.32 |
| Fluor Protector*) | 10.36 | 0.35 | 0.58 | 0.09 | 39.79 | 0.20 | 0.00 | 48.64 |
| Duraphat*) | 6.59 | 2.39 | 1.70 | 0.28 | 40.04 | 0.00 | 0.00 | 49.00 |
| MI Varnish*) | 5.69 | 0.57 | 1.33 | 0.31 | 41.02 | 0.00 | 0.00 | 51.08 |
| Clinpro White Varnish*) | 5.20 | 0.24 | 0.93 | 0.26 | 41.99 | 0.00 | 0.00 | 51.38 |
| Profluorid Varnish*) | 5.94 | 0.19 | 0.77 | 0.16 | 41.46 | 0.00 | 0.00 | 51.47 |
| Untreated enamel*) | 6.07 | 0.15 | 0.86 | 0.14 | 41.99 | 0.00 | 0.00 | 50.77 |

*)Comparative example

### Example 4

In Vitro Fluoridation of Enamel by Fluoride Varnishes (1 h)

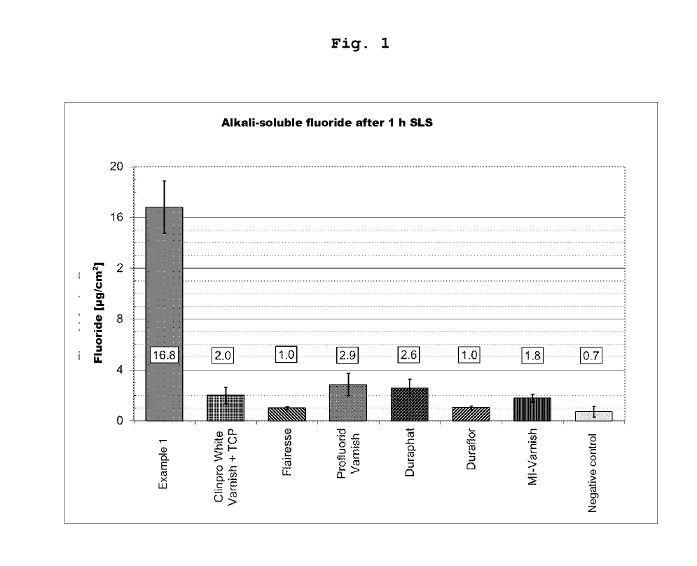

Analogously to Example 2, enamel blocks from bovine teeth were treated with fluoride-containing varnishes and the alkali-soluble fluoride (ASF) was measured. The dried samples were stored in artificial saliva (saliva-like solution, abbr.: SLS) at 37° C. for only 1 hour. The results are shown in Table 4 and represented in graphical form in FIG. 1.

TABLE 4
Results of the in vitro fluoridation of enamel after 1 h of treatment

| Product | Manufacturer | ASF [μg/cm²] |
|---|---|---|
| Example 1 | | 16.80 ± 2.06 |
| Clinpro White Varnish + TCP*) | (3M-ESPE, Seefeld, DE) | 2.00 ± 0.66 |
| Flairesse*) | (DMG, Hamburg, DE) | 1.00 ± 0.11 |
| Profluorid Varnish*) | (VOCO, Cuxhaven, DE) | 2.86 ± 0.89 |
| Duraphat*) | (Colgate, USA) | 2.58 ± 0.68 |
| Duraflor*) | (Medicom, Lachine, CAN) | 1.03 ± 0.14 |
| MI Varnish *) | (GC Corp., Tokyo, J) | 1.78 ± 0.32 |
| Negative control | | 0.72 ± 0.42 |

*)Comparative example

### Example 5

Preparation of a Fluoride-Containing Varnish Without Plasticizer

A varnish having the following composition was prepared by mixing the components:

| Proportion | Component |
|---|---|
| 57.90 wt. % | ethanol |
| 14.48 wt. % | water |
| 25.00 wt. % | terpolymer of tert-butyl acrylate, |
| | ethyl acrylate and methacrylic acid |
| | (INCI: acrylates copolymer) |
| 0.02 wt. % | saccharin sodium |
| 2.0 wt. % | ammonium fluoride |
| 0.50 wt. % | pyrogenic aluminium oxide |
| 0.10 wt. % | peppermint flavouring |

All the constituents were dissolved, accompanied by stirring, in the solvent introduced. A stable, practically colourless, homogeneous, low-viscosity varnish was obtained.

The varnish was applied to enamel blocks from bovine teeth as described in Example 4 and alkali-soluble fluoride (ASF) was measured after storage in artificial saliva (ASF) for 1 h. A value of 29.09±3.22 μg/cm² was found (negative control (deion. water): 0.68±0.03 μg/cm2).

1. Fluoride-containing varnish for application to the tooth surface, characterized in that it contains
25-87.5 wt. % organic solvent,
2-50 wt. % water,
5-50 wt. % water-insoluble film-forming agent,
0.5-10 wt. % inorganic fluoride source in dissolved form,
in each case relative to the total weight of the varnish.

2. Fluoride-containing varnish according to claim 1, which additionally contains 5 to 25 wt. % plasticizer.

3. Fluoride-containing varnish according to claim 2, which contains, as the plasticizer, fatty alcohol, polyethylene glycol (PEG), polypropylene glycol (PPG), dexpanthenol, an ester or a mixture thereof.

4. Fluoride-containing varnish according to claim 3, which contains, as the plasticizer, a sugar ester or an alkyl or phenyl ester of di- or tricarboxylic acids or hydroxydi- or -tricarboxylic acids having one or more hydroxyl groups.

5. Fluoride-containing varnish according to claim 4, which contains, as the sugar ester, an ester of an organic acid with a mono- or disaccharide.

6. Fluoride-containing varnish according to claim 5, which contains, as the sugar ester, an ester of sucrose with acetic acid or isobutyric acid or a mixed ester of sucrose with acetic acid and isobutyric acid.

7. Fluoride-containing varnish according to claim 1, which contains, as the organic solvent, a water-miscible mono- or polyvalent alcohol and/or a water-miscible ketone.

8. Fluoride-containing varnish according to claim 1, which contains an inorganic fluoride source which has a solubility in water at 25° C. of at least 5 mol in 1 kg water.

9. Fluoride-containing varnish according to claim 8, which contains, as the inorganic fluoride source, NH₄F, KF, RbF, CsF, NH₄HF₂, KHF₂ or a mixture thereof.

10. Fluoride-containing varnish according to claim 1, which contains, as the film-forming agent, an alcohol- or ketone-soluble polymer having a solubility in water of at most 5.0 wt. %.

11. Fluoride-containing varnish according to claim 10, which contains, as the film-forming agent, an acrylate and methacrylate homopolymer, acrylate and methacrylate copolymer, a terpolymer of C₄-C₈ alkylacrylamide, acrylates and acrylic acid, a terpolymer of methacrylic acid and acrylates, a vinyl acetate/crotonic acid copolymer, a terpolymer of tert-butyl acrylate, ethyl acrylate and methacrylic acid (INCI name: acrylates copolymer) or a mixture thereof.

12. Fluoride-containing varnish according to claim 1, which contains
50-70 wt. % ethanol,
10-20 wt. % water,
5-15 wt. % terpolymer of tert-butyl acrylate, ethyl acrylate and methacrylic acid,
10-20 wt. % sucrose acetate isobutyrate,
1-2 wt. % ammonium fluoride,
0.05-0.2 wt. % flavouring substance(s) and
0.01 to 0.1 wt. % sweetening agent.

13. Fluoride-containing varnish according to claim 1, which additionally contains 0.01-10 wt. % filler and/or antibacterial active ingredients.

14. Fluoride-containing varnish according to claim 1, which has a viscosity of less than 5,000 mPa·s (measured at 15.0° C. and a shear rate of 100 s⁻¹).

15. A method of treating hypersensitive teeth and/or tooth necks comprising applying the fluoride-containing varnish according to claim 1, to a tooth or teeth in the prophylaxis of caries, in the treatment of incipient caries lesions, the inhibition of demineralization and/or tooth erosion and in the fluoridation of enamel.

16. Fluoride-containing varnish according to claim 1, which after a treatment time of one hour produces a fluoridation of enamel (ASF) of at least 10 μg/cm² measured on bovine tooth enamel.

17. Fluoride-containing varnish according to claim 1, which forms a colourless, clear varnish film after drying.

18. Fluoride-containing varnish according to claim 6, wherein the mixed ester of sucrose with acetic acid and isobutyric acid comprises sucrose acetate isobutyrate.

19. Fluoride-containing varnish according to claim 7, wherein the organic solvent comprises isopropanol, acetone, or ethanol.

20. Fluoride-containing varnish according to claim 8, wherein the inorganic fluoride source comprises a solubility in water at 25° C. of at least 10 mol in 1 kg water.

21. Fluoride-containing varnish according to claim 14, which has a viscosity of less than 1,000 mPa·s (measured at 15.0° C. and a shear rate of 100 s⁻¹).

22. Fluoride-containing varnish according to claim 21, which has a viscosity of less than 250 mPa·s (measured at 15.0° C. and a shear rate of 100 s⁻¹).

23. Fluoride-containing varnish according to claim 16, which after a treatment time of one hour produces a fluoridation of enamel (ASF) of at least 15 μg/cm², measured on bovine tooth enamel.
